(19) United States
(12) Patent Application Publication
(10) Pub. No.: US 2023/0320917 A1
(43) Pub. Date: Oct. 12, 2023

(54) VERTICALIZING TABLE EQUIPPED WITH MEANS OF MOBILIZATION OF THE LOWER LIMBS AND LIGHT SOURCES

(71) Applicant: PANDHORA S.R.L., Napoli (IT)

(72) Inventors: Stefano Troncone, Salerno (IT); Alexander Troncone, Salerno (IT); Chrystel Troncone, Salerno (IT)

(73) Assignee: PANDHORA S.R.L., Napoli (IT)

(21) Appl. No.: 18/027,062

(22) PCT Filed: Sep. 17, 2021
(86) PCT No.: PCT/IB2021/058502

§ 371 Date: Mar. 17, 2023

(30) Foreign Application Priority Data
Sep. 21, 2020 (IT) 102020000022195

(51) Int. Cl. A61H 1/02; A61N 5/06

(52) U.S. Cl. CPC A61H 1/0262; A61N 5/0625; A61H 2201/10; A61H 2201/0142; A61H 2203/0481; A61N 2005/0659; A61H 2201/1642; A61H 2201/5007; A61H 2201/1246; A61H 2201/123; A61H 2201/1676; A61H 2201/1652

(57) ABSTRACT

Mobility rehabilitation device comprising a table, hinged to a support structure and respective actuator, configured to rotate said table between a horizontal and a vertical position, said table being provided with a harness configured to fasten a patient positioned thereon, said rehabilitation device comprising a lower limbs movement mechanism to make the patient's femur rotate with respect to the hip and a couple of feet movement platforms, said device comprising also a plurality of infrared light sources, positioned so that each light source lights up the muscles of a respective area of the patient's lower limbs, a control unit configured to control said lower limbs movement mechanism and said light sources and to manage an adjusting cycle of the intensity of said light sources in a differentiated manner for each of said sources, as a function of the movement cycle imposed by said lower limbs movement mechanism.

### BACKGROUND OF THE INVENTION

### 1. Field of the Invention

The present invention relates to a robot equipment for rehabilitation. In particular, the present invention relates to a verticalization table provided with lower limbs mobilization means and sources of infrared radiation.

Robotic rehabilitation allows to regain an almost normal life even after traumas and invalidating pathologies. In fact, the usage of robots for physiotherapy has opened up new horizons in the treatment of permanent and temporary disability. These are in fact robotic systems for limbs re-education.

### 2. Brief Description of the Prior Art

In particular, devices exist for robotic rehabilitation made up of verticalization tables, which can be moved from a horizontal position to a vertical position, on which a patient is laid and fastened by suitable harnesses. Then the table the patient is laid on is brought gradually from the horizontal to the vertical position, and dedicated kinematisms make the patient carry out movements with the lower limbs.

Verticalization tables provided with robotized steppers are known, as for example the ones described in Patents EP2730265 and EP1169003. The functioning is the following: when the patient is verticalized the steppers provide a mobilization of the lower limbs which can occur both actively (i. e. with the patient participating to the muscle effort) and passively.

The machine moves the lower limbs with a programmed speed according to a scheme of alternated flexions and extensions, thus stimulating the active participation of the patient according to his capacity of collaboration.

Generally, in all the known devices the upper portion of the body is held to the tilting table by means of a harness fastening the patient at his chest and shoulders.

The thighs of the patient are fixed to suitable movement mechanisms and the feet are fastened to suitable movable plates provided with springs.

Consequently, the flexion and extension movements of the hips caused by the movement mechanisms impose also a loading and discharging action of the bearing force on the feet. In this way, the therapy influences the movements of the joints of hips, knees and ankles. The mobilization sequence of the lower limbs is controlled by a computer which allows to adjust the movement of the legs, the load, the speed, the extension of the hip and other parameters. The advantage of these devices is that they allow to carry out a fast and intensive therapy.

In order to increase the proprioceptive stimulation of the patient (i. e. his awareness of the movement he is carrying out) in the devices known at the state of the art monitors are used, positioned so that the patient can see them, which reproduce as virtual reality the movements he is carrying out.

In this way, the therapy becomes even more effective since the patient is able to associate mentally the movement he is carrying out to the sensations of his own legs.

Anyway, the perception of the patient of the movement he is carrying out is not of physical type but exclusively visual. Moreover, verticalization tables exist provided with robotized steppers and devices configured to apply electrical stimulations to the patient according to a scheme associated to the movement he is carrying out, in order to maximize the therapy by acting in this manner on the neuronal plasticity. These systems apply the integrated functional electric stimulation (FES) which increases the effects of the early verticalization on the cardiovascular system.

Anyway, this kind of stimulation is very invasive, since it needs electrodes to be installed on the patient's legs, which make electric current pass through the muscle in order to stimulate it. For many subjects, for example children, the electric stimulation can be uncomfortable. An electric stimulation of the leg muscles can be not tolerated.

At the best of the current inventors' knowledge, there are not known devices of the just described type which implement technical solutions intended to optimize the effectiveness of the rehabilitation treatment, and in particular to improve the physical perception of the movement the user is carrying out.

In other technical fields, there are also known systems combining the infrared radiation with muscle movements, for training or for improving the loosing of weight in determined body parts. U.S. Pat. No. 6,024,760 describes a device to reduce adipose tissue in determined areas by stimulating lipolysis through the heat provided by generators of infrared rays while the user carries out fitness exercises or general muscle activity.

WO212148064A1 describes a fitness tool for abdominal friction exercises for abdominal loosing of weight comprising an infrared lamp directed to the portion of the abdomen object of friction.

US2009054217 describes a tiltable inversion fitness tool which comprises a generator of infrared rays to facilitate or favor blood circulation.

So, there are generally known techniques providing the usage of infrared rays to improve blood circulation and for muscle relaxation, but there are not known techniques where the infrared stimulation is used to improve the perception a user has of his own body and movements he is carrying out.

### SUMMARY OF THE INVENTION

Therefore, aim of the present invention is to provide a verticalization table which overcomes the limits linked to the embodiments known at the state of the art, and which optimizes the effectiveness of the treatment and the perception each user has of the movement he is carrying out.

According to an aim the present invention is intended to facilitate the post-traumatic rehabilitation by using infrared radiations combined with the muscle movement obtained by means of a verticalization table.

The present invention realizes the prefixed aims since it is a device for mobility rehabilitation comprising a table (2), hinged at an end to a support structure (3) and respective actuator (4), configured to make said table (2) rotate between a horizontal position and a vertical position, said table (2) being provided with a harness (15) configured to fasten a patient positioned thereon so that the patient cannot fall down even when the table is in vertical position, said rehabilitation device comprising also a lower limbs movement mechanism (8) configured to make the patient's femur rotate with respect to the hip and a couple of feet movement platforms (6), characterized in that said device comprises also a plurality of infrared light sources (11), fastened to respective supports (10) integral to said support structure (3) and positioned so that each light source lights up the muscles of a respective area of the patient's lower limbs, a data processing and control unit (13) configured to control said lower limbs movement mechanism (8) and said light sources (11) and in that said data processing and control unit (13) is configured also to manage an adjusting cycle of the intensity of said light sources (11) in a differentiated manner for each of said sources, as a function of the movement cycle imposed by said couple of mobilization actuators of the lower limbs.

### BRIEF DESCRIPTION OF THE DRAWINGS

The present invention is described in the following with reference to the appended figures:

FIG. 1 shows a perspective view of the invention with all its essential elements and in which the arrows indicate the possible movements of some components of the device.

FIG. 2 shows a side view of the invention illustrating the table (2) in almost vertical position with the data processing and control unit (13) managing an adjusting cycle of the intensity of said light sources (11), in a differentiated manner for each of said sources, as a function of the movement cycle imposed by said couple of lower limbs mobilization actuators (8).

The arrows indicate the movement induced by the lower limbs mobilization actuators (8).

FIG. 3 shows a front view of the invention in which the light sources (11) irradiate only the left leg of the patient corresponding at the same time in which it is moved by the left actuator.

FIG. 4 shows a side perspective view of an application of the invention illustrating the functioning of the invention in the configuration with verticalization table (2) in horizontal position.

FIG. 5 shows a schematic indication of the light intensity variation cycles.

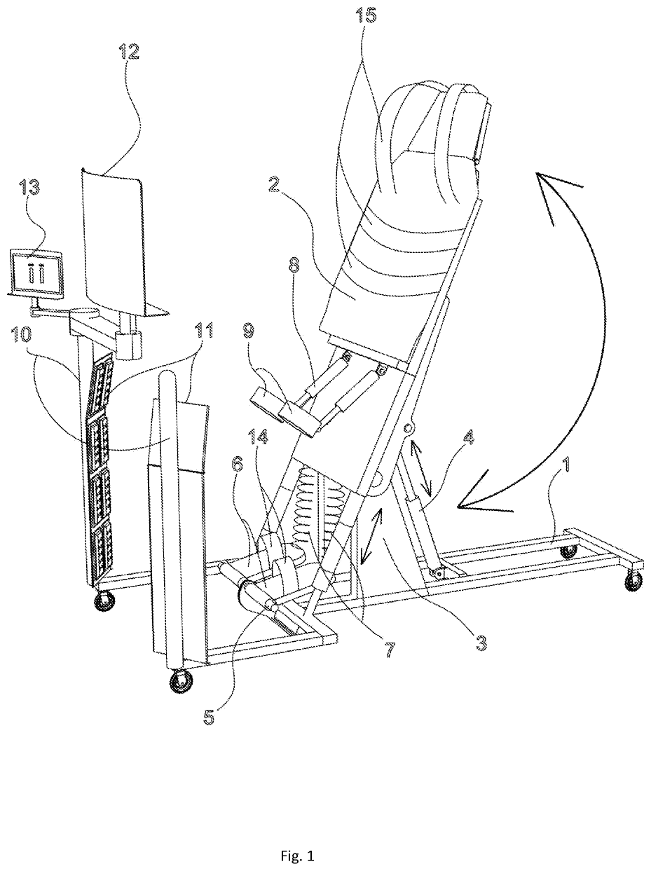

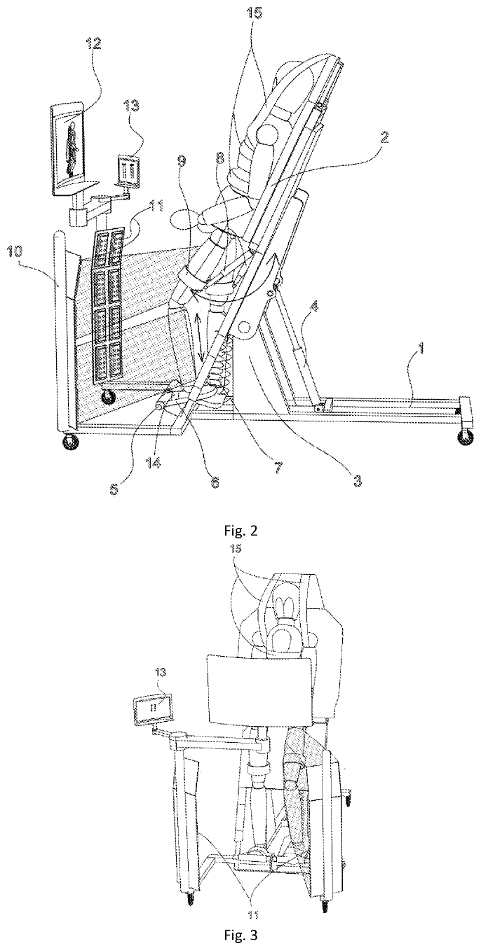

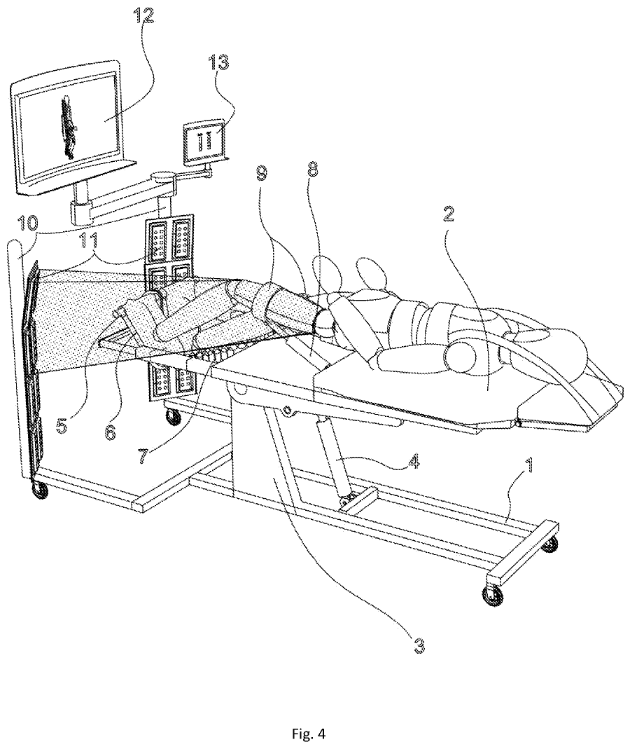

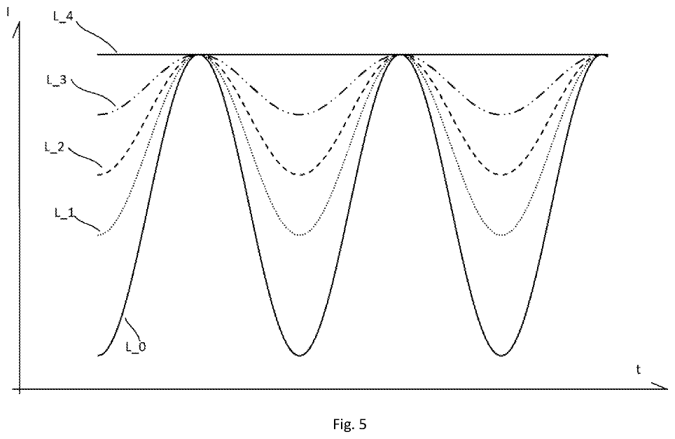

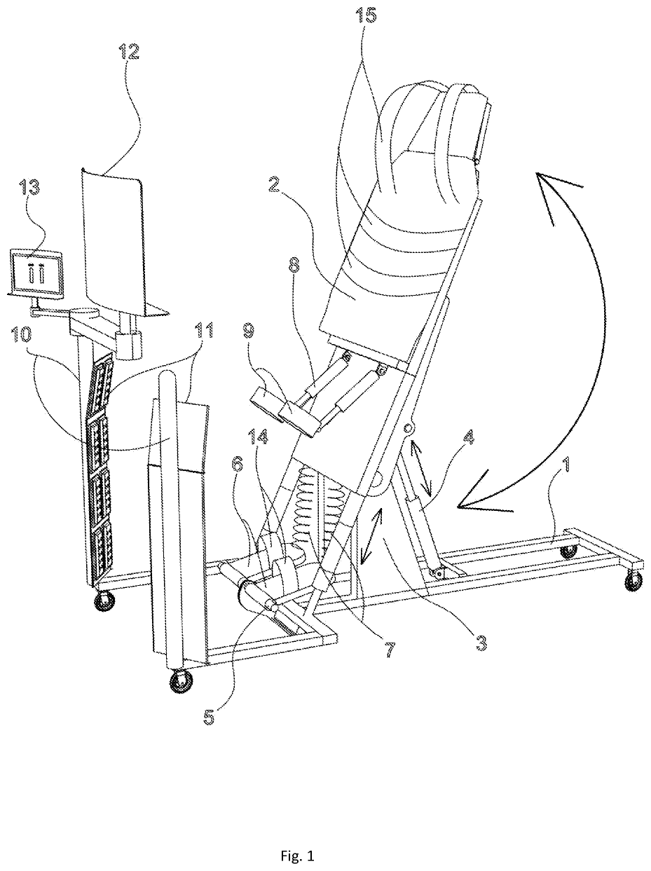

With reference to what shown in the appended figures, the device according to the invention comprises a table (2), hinged at an end to a support structure (3) integral to a base (1) on rollers and a respective actuator (4), configured to make said table (2) rotate between a horizontal position and a vertical position, said table being configured to take stably any position between the horizontal and the vertical one.

### DETAILED DESCRIPTION OF THE PREFERRED EMBODIMENTS

In a preferred and not limiting embodiment, said actuator (4) is a hydraulic or electric piston hinged at an end to the base (1), and at the other end to the table (2).

The table (2) is also provided with a harness (15) configured to fasten the patient's chest and shoulders so that the patient cannot fall down even when the table is in vertical position.

The device comprises also a lower limbs movement mechanism (8).

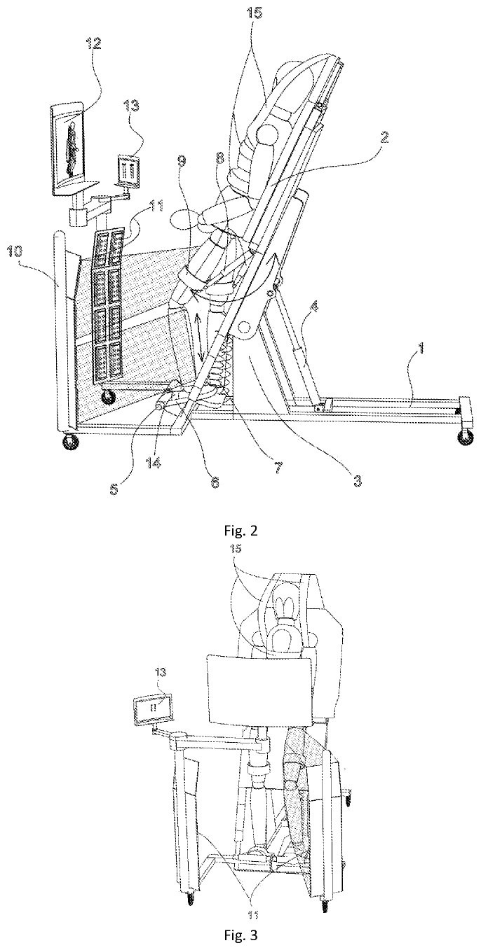

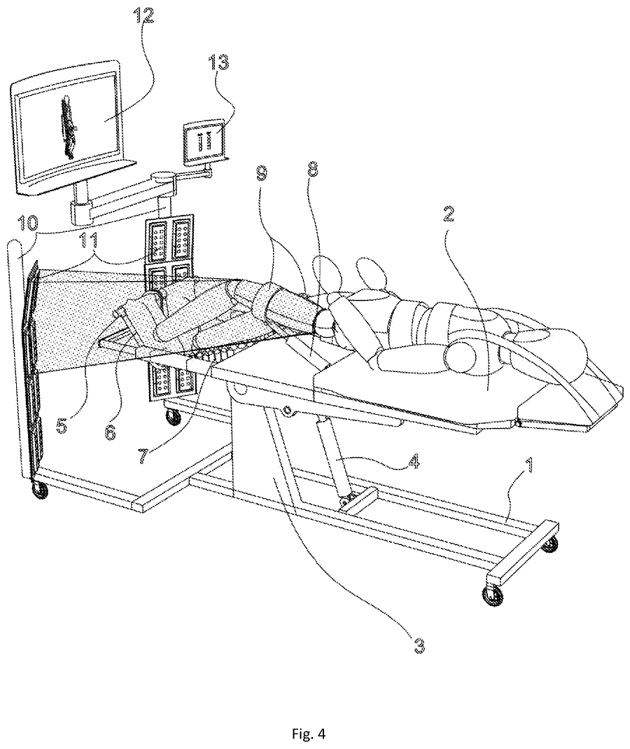

In an embodiment, said mechanism comprises a couple of linear actuators, of electric or hydraulic type, each hinged at an end to the table (2) at the position of the hip of each leg of the patient and at the opposite end to a respective fastening means (9) for the patient's femur. Preferably, the fastening means (9) comprises a semicircular rigid support inside which the patient's femur can be fastened by means of a band, when the patient lays on the verticalization table (2). From the just described geometry it is clear that according to what shown for example in FIGS. 2 and 4, a rotation movement of the patient's femur with respect to the hip corresponds to an extension of each one of said actuators (8).

Generally, the table comprises a kinematism and respective movement means configured to make the patient's femur rotate with respect to the hip, so that it is possible to control the movements of the lower limbs of the patient.

The device comprises also a couple of feet movement platforms (6) positioned at the position of the patient's feet, each hinged with its own front end to an axis rotation (5).

In a first embodiment, said axis of rotation (5) is integral to the verticalization table (2); in a second embodiment said axis is fastened so that it can translate according to a parallel direction to said table (2), so that the foot can carry out a roto-translation movement as a whole.

Each platform (6) is also provided with a spring (7) or other elastic pushing means configured to push upwards the rear portion of said platform (6) making it rotate around its own axis of rotation.

It is clear that an elongation or compression of the respective pushing means corresponds to a rotation of each platform (6) around its own axis of rotation positioned at the front end.

The springs (7) allow the platforms (6) to support the foot during the execution of the whole movement of the leg induced by the actuator (8).

The device comprises also a plurality of infrared light sources (11), fastened to respective supports (10) integral to the base (1), and positioned so that each light source lights up the muscle of a respective area of the patient's lower limbs, and a data processing and control unit (13) provided with a display (12) positioned so that the user can see it and configured to control said lower limbs movement mechanism (8) and said light sources (11). The device is characterized in that said data processing and control unit (13) is configured to manage an adjusting cycle of the intensity of said light sources (11), in a differentiated manner for each of said sources, as a function of the movement cycle imposed by said couple of lower limbs mobilization actuators.

In a preferred embodiment, on said processing and control unit (13) computer programs are stored configured to implement the following method:
1) subdividing the lower limbs in a plurality of areas, each area being associated to the stimulation by means of a respective infrared light source (11);
2) for each of said areas defined at point 1, identifying a main muscle group and schematizing the action cycle imposed by said couple of lower limbs mobilization actuators (8) as a sequence of a plurality of contraction and relaxation steps of said main muscle group;
3) determining, for each of said light sources (11) associated to said areas, an adjusting cycle of the intensity of the infrared radiation emitted as a function of said contraction and relaxation cycle schematized at point (2) for the area associated to the specific light source.

The adjusting cycle of the intensity is preferably configured so that each light source has a maximum emission intensity at the contraction steps of the respective muscle group and a minimum emission intensity at the relaxation steps of the respective muscle group.

Each light source can be possibly switched on with a predetermined advance of time with respect to the contraction step of the respective muscle group.

In another embodiment, the device is characterized in that the movement mechanism (8) comprises means for measuring the power supplied in each moment during the execution of the movement.

This measure allows to define a profile of time variation of the power supplied by the device during the execution of each movement.

In this case, the intensity adjusting cycle can be conveniently configured so that the intensity of each of said light sources (11) oscillates cyclically between a base value and a maximum value, and said base value is obtained as the sum of a minimum threshold value and a value proportional in each moment to the power provided as contribution to the movement by the patient.

The power provided by the patient can be calculated as the difference between the value of power supplied by the mechanism in each moment of a movement cycle in which the patient remains still with his own muscles and the power supplied by the mechanism in the same moment as the current cycle.

Such difference indicates in fact the muscle effort applied by the patient.

In this manner, a feedback physical stimulation is provided to the patient, indicating in each moment: the muscle groups in contraction step; the level of applied muscle effort.

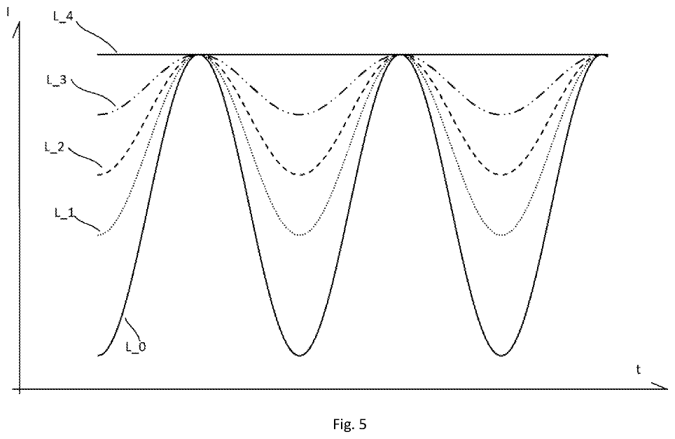

An example of such intensity adjusting cycle, for various effort levels applied by the patient, is shown in the appended FIG. 5.

At a first effort intensity level (L_0) in which the patient does not contribute in any way to the movement, the intensity (I) of each light source is adjusted so that it varies cyclically, during the movement, between the minimum threshold value and the maximum value, and is synchronized so that the maximum value coincides with the contraction moment of the respective muscle group.

While the effort of the patient increases (level L_1), the base value results from the sum of the minimum threshold value with the value proportional to the muscle effort exerted by the patient, and so it is higher than the base value in case of absence of effort of the patient (L_0).

At another and progressive increase of the effort applied by the patient (L_2, L_3), such base vale increases consequently, up to reach the condition (L_4) in which the patient is able to carry out the movement only relying on his own forces, without the need of an intervention of the machine, and in this case the light radiation intensity is constant and equal to the maximum value.

Preferably, said areas defined at point (1) comprise, for each side of the patient, an area associated to the gluteus, an area associated to the front femoral portion, an area associated to the rear femoral portion, an area associated to the front tibial portion, an area associated to the rear tibial portion.

The couple of lower limbs mobilization actuators (8) can be of passive type.

A display (12), which can be seen by the patient and which reproduces the movement of the patient, can be installed on the base (1).

This synchrony between the thermal stimulations and the walking cycle amplifies the perception the user has of the movement he is carrying out, and speeds up the rehabilitation cycle. Moreover, mild heat is well tolerated and makes the therapy more comfortable for the patient.

In another embodiment, the light radiation intensity can be function of the position of said platforms (6), since the position of said platforms indicates the force applied by the patient to compress the pushing elastic means associated to each platform. It is to be specified also that said platforms (6), preferably but not limitingly, can be provided with automatic movement means configured to make the patient's foot carry out movement cycles which simulate the walking movement, and which comprise a series of extensions and flexions of the foot.

Moreover, the movement means of said platforms are preferably provided with measuring means of the mechanical power supplied in order to make the patient's foot carry out the just described movement.

In such case, the device according to the invention can be configured so that the light sources intensity oscillates cyclically between a base value and a maximum value, and said base value is obtained as the sum of a minimum threshold value and a value proportional in each moment to the power provided as contribution to the movement by the patient and calculated as the difference between the value of power supplied by the movement means of the platforms in a movement cycle in which the patient remains still and the power supplied by the same movements means in the same moment as the current cycle. Such difference is in fact indicative of the muscle effort applied by the patient.

In a preferred embodiment, the device according to the invention is configured to carry out the following method:

(100) Heating of the Patient

Before carrying out the therapy by means of the machine, the patient is subjected to heating for a time interval between 2 and 5 minutes by means of the provided infrared light sources, in order to produce a general dilation of the blood vessels, with consequent increase of the blood circulation and activation of the metabolism of tissues and cells.

So, by favoring the oxygenation of the muscles and their elasticity, the heating allows to avoid contractures or strains, above all in patients whose limb subjected to the therapy has lost its own functionality. The heat emitted by the infrared rays heats the muscle, thus having a lenitive action which reduces the stress and renders the joints more flexible.

(200) Mobilization of Lower Limbs and Contemporary Heating of the Patient

During this step, the intensity of the infrared rays is adjusted so to follow proportionally the intensity of the effort of each muscle group, according to what above described.

While increasing proportionally with the effort of the patient, the heat stimulates the thermoreceptors provided on the skin. Thermoreceptors are receptors sensible to heat and cold which are different from the other sensorial receptors on the skin since they are always active, so to inform the brain constantly about the temperature variation during a cycle imposed by the machine.

By visually observing and perceiving the intensity variation of the infrared rays as a function of the effort made during the movement, the patient will be able to evaluate by himself instantly the success of the rehabilitation.

This will allow the patient to control autonomously his own activity, thus being more involved in the therapy and thus being motivated for improvement.

In this way the patient can interact with the intensity of irradiation according to the effort made, by connecting sensorially to the machine, maximizing the biofeedback.

As yet said, the intensity of the radiation will be proportional to the effort applied by the patient, calculated as the difference between the power needed to move the patient's legs with no collaboration of the same (measured in an initialization cycle of the treatment) and the power supplied during each following cycle.

Such parameter is used to check that the patient works during rehabilitation or training.

In fact, the less power is supplied by the device, the higher the effort will be that the patient applies to carry out the movement.

This makes the power supplied a further instrument able to stimulate the patient: by decreasing such power, the patient has necessarily to participate actively, thus increasing the work he has to carry out. When the conduction force is deactivated, the movement will be exerted only by the patient able to keep a model of physiological model.

1. A device for mobility rehabilitation comprising a table (2), hinged at an end to a support structure (3) and respective actuator (4), configured to make said table (2) rotate between a horizontal position and a vertical position, said table (2) being provided with a harness (15) configured to fasten a patient positioned thereon so that the patient cannot fall down even when the table is in vertical position,
said rehabilitation device comprising also a lower limbs movement mechanism (8) configured to make the patient's femur rotate with respect to the hip and a couple of feet movement platforms (6), characterized in that said device comprises also a plurality of infrared light sources (11), fastened to respective supports (10) integral to said support structure (3) and positioned so that each light source lights up the muscles of a respective area of the patient's lower limbs,
a data processing and control unit (13) configured to control said lower limbs movement mechanism (8) and said light sources (11), and in that said data processing and control unit (13) is configured also to manage an adjusting cycle of the intensity of said light sources (11) in a differentiated manner for each of said sources, as a function of the movement cycle imposed by said couple of mobilization actuators of the lower limbs.

2. The device according to claim 1, wherein said processing and control unit (13) computer programs are stored configured to implement the following steps:
1) subdividing the lower limbs in a plurality of areas, each infrared light source (11) being configured to stimulate a respective area;
2) for each of said areas defined at step 1, identifying a main muscle group and schematizing the action cycle imposed by said lower limbs mobilization movement mechanism (8) as a sequence of a plurality of contraction and relaxation steps of said main muscle group;
3) determining, for each of said light sources (11) associated to said areas, an adjusting cycle of the intensity of the infrared radiation emitted as a function of said contraction and relaxation cycle schematized at point (2) for the area associated to the specific light source.

3. The device according to claim 2, wherein said adjusting cycle of the intensity is configured so that each light source has a maximum emission intensity at the contraction steps of the respective muscle group and a minimum emission intensity at the relaxation steps of the respective muscle group.

4. The device according to claim 1, wherein said movement mechanism (8) comprises control means for measuring the power supplied in each moment during the execution of the movement.

5. The device according to claim 4, wherein said control means are configured to calculate the power provided by the patient as the difference between the value of power supplied by the mechanism in any moment of a movement cycle in which the patient remains still and the power supplied by the mechanism in the same moment as the current cycle.

6. The device according to claim 1, wherein said adjusting cycle of intensity can be configured so that the intensity of each of said light sources (11) oscillates cyclically between a base value and a maximum value, said base value being obtained as the sum of a minimum threshold value and a value proportional in each moment to the power provided as contribution to the movement by the patient.

7. The device according to claim 2, wherein said areas defined at step (1) comprise, for each side of the patient, an area associated to the gluteus, an area associated to the front femoral portion, an area associated to the rear femoral portion, an area associated to the front tibial portion, an area associated to the rear tibial portion.

8. The device according to claim 1, wherein said support structure (3) in integral to a base (1) on rollers.

9. The device according to claim 1, wherein said table (2) is configured to take stably any position between the horizontal and the vertical one.

10. The device according to claim 1, wherein said actuator (4) is a hydraulic or electric piston hinged at an end to the base (1), and at the other end to the table (2).

11. The device according to claim 1, wherein said movement mechanism (8) comprises a couple of linear actuators, of electric or hydraulic type, each hinged at an end to the table (2) at the position of the hip of each leg of the patient and at the opposite end to a respective fastening means (9) for the patient's femur.

12. The device according to claim 1, wherein said platforms (6) are hinged each with their own front end to the axis of rotation (5) integral to said table (2), and each of said platforms (6) comprises also an elastic pushing means configured to push upwards the rear portion of said platform (6) making it rotate around its own axis of rotation.

13. The device according to claim 1, wherein said platforms (6) are hinged each with their own front end to an axis of rotation (5) fastened so that it can translate according to a direction parallel to said table (2), and each of said platforms (6) comprises also an elastic pushing means configured to push upwards the rear portion of said platform (6) making it rotate around its own axis of rotation.

14. The device according to claim 1, wherein the intensity of the light radiation of at least one of said light sources (11) is a function of the position of said platforms (6).

15. The device according to claim 1, further comprising automatic movement means of said platforms, configured to make the patient's foot carry out movement cycles which simulate the walking movement, and measuring means of the mechanical power supplied by said platform movement means, and characterized in that the light sources intensity of at least one of said light sources oscillates cyclically between a base value and a maximum value, said value being obtained as the sum of a minimum threshold value and a value proportional to the difference between the value of power supplied by the movement means of the platforms in a movement cycle in which the patient remains still and the power supplied by the same movements means in the same moment as the current cycle.
